(19) United States
(12) Patent Application Publication
(10) Pub. No.: US 2025/0359896 A1
(43) Pub. Date: Nov. 27, 2025

(54) RADIALLY EXPANDABLE BONE SCREW

(71) Applicants: Shawn P. Fojtik, Park City, UT (US); Cortny Robison, Salt Lake City, UT (US)

(72) Inventors: Shawn P. Fojtik, Park City, UT (US); Cortny Robison, Salt Lake City, UT (US)

(21) Appl. No.: 19/180,105

(22) Filed: Apr. 15, 2025

Related U.S. Application Data
(60) Provisional application No. 63/634,323, filed on Apr. 15, 2024.

(51) Int. Cl. A61B 17/70; A61B 17/56; A61B 17/86

(52) U.S. Cl. CPC A61B 17/7032; A61B 17/8635; A61B 17/864; A61B 17/8685; A61B 2017/564; A61B 2017/8655

(57) ABSTRACT

A bone screw includes an expandable section with struts that expand and twist to engage bone and, thus, to secure the orthopedic screw within the bone. Expansion of the expandable section may occur as the expandable section is axially or longitudinally compressed. Such compression may be effected with an interior element of the bone screw and, optionally, with a tool. Methods for implanting bone screws are also disclosed.

### CROSS-REFERENCE TO RELATED APPLICATION

A claim for priority to the Apr. 15, 2024 filing date of U.S. Provisional Patent Application No. 63/634,323, titled SURGICAL ORTHOPEDIC SCREW WITH OUTWARDLY EXPANDING STRUTS (“the '323 Provisional Application”) is hereby made pursuant to 35 U.S.C. § 119(e). The entire disclosure of the '323 Provisional Application is hereby incorporated herein.

### TECHNICAL FIELD

This disclosure relates generally to orthopedic screws, or bone screws. More specifically, this disclosure relates to orthopedic screws with struts that expand and twist to engage bone and, thus, to secure the orthopedic screws within bone. In addition, this disclosure relates to methods for using orthopedic screws alone and to secure other orthopedic implants to bone.

### RELATED ART

Bone screws are commonly used to facilitate the healing of fractures and to stabilize bone. Bone screws may be used alone or to secure other orthopedic implants, such as rods, plates, and other hardware, to bone. A conventional bone screw typically pulls fragments of fractured bone together, which is known as compression.

A pedicle screw is a type of bone screw that may be used with a rod to secure vertebra together in spinal fusion surgeries. Spinal fusion surgeries are performed to stabilize and support the spine when subject to a variety of conditions, including conditions that cause instability, pain, or deformity. Some examples of spinal conditions that may be treated with spinal fusion surgery, including degenerative disk disease, herniated discs, spinal stenosis, spinal fractures, scoliosis, spondylolisthesis, and spinal tumors. The market for spinal surgery devices has seen substantial growth due to technological advancements, an aging population, and an increasing prevalence of spinal conditions.

Despite widespread use of pedicle screws and the advancements in surgical techniques and implant design, the efficacy of pedicle screws is significantly challenged by the rate of loosening, a complication that can lead to poor clinical outcomes, additional surgeries, and increased healthcare costs. When spinal fusion surgery is approached posteriorly, two pedicle screws are typically inserted into each of the fused vertebra. The positions and orientations the pedicle screws are introduced into the vertebra may be determined by the hardware (e.g., the hardware with which the pedicle screw is used), the patient's anatomy, and/or the surgeon. In any event, hardware, or surgeon dependent but overall positioning involves insertion of a screw through the pedicle and into the vertebral body. Like other types of bone, vertebra typically include more compact cortical bone on the outside (i.e., next to the surface) and less dense cancellous bone internally. While bicortical approaches (i.e., through the cortical bone on a proximal surface, the internal cancellous bone, and the bicortical bone on an opposite, distal surface) are typically recommended for other types of bone screws to ensure that the bone screws are securely anchored within the bone, human anatomy (e.g., the locations of the aorta, vena cava, liver, intestines, etc.) makes the use of bicortical approaches unacceptably risky for posteriorly approached spinal fusions, which may adversely affect the extent to which pedicle screws can anchor into the vertebra.

The loosening of pedicle screws and other bone screws is particularly problematic in patients with compromised bone quality, including patients who suffer from osteopenia or osteoporosis. Osteoporosis, a condition characterized by decreased bone mineral density and increased fracture risk, is prevalent in an estimated 500 million people worldwide. Ganesan K, et al., Secondary Osteoporosis (StatPearls Publishing, 2025) (https://www.ncbi.nlm.nih.gov/books/NBK470166/). In the context of spinal surgery, osteoporotic bone presents a challenge for pedicle screw fixation due to the diminished bone mass, which compromises the screw's hold and stability. Studies indicate that the rate of pedicle screw loosening in healthy patients ranges from <1% to 15%, but this rate escalates significantly in patients with osteoporosis, with reported loosening rates as high as 60%. Yuan L, et al., “Incidence, Risk, and Outcome of Pedicle Screw Loosening in Degenerative Lumbar Scoliosis Patients Undergoing Long-Segment Fusion,” Global Spine J 13(4):1064-1071 (2023).

Similar issues are also a prevalent issue in a variety of other conditions where bone screws are used, including the repair of proximal humerus fractures (up to 23% screw failure incidence) (Burkhard B, et al., “Overdrilling increases the risk of screw perforation in locked plating of complex proximal humeral fractures-A biomechanical cadaveric study,” J Biomech 117:110268 (Mar. 5, 2021)), hip fractures (up to 10% screw failure incidence) (Sun H, et al., “Decreased complications but a distinctive fixation loosening mechanism of fully threaded headless cannulated screw fixation for femoral neck fractures in young adults,” J Orthop Surg Res 6(1):234 (Mar. 30, 2021)), and pelvic fractures (up to 20% screw failure incidence) (Zhou W, et al., “Incidence of and Risk Factors for Screw Loosening after Iliosacral Screw Fixation for Posterior Pelvic Ring Injury,” Orthop Surg (Jul. 15, 2023)).

### SUMMARY

A bone screw of this disclosure may include an expandable element with struts. The expandable element of the bone screw may be shortened, which may exert a radial force (i.e., compression) and a twisting force (i.e., torsion) on the struts. The struts may, in turn, exert compressive and torsional forces on bone into which the bone screw is inserted.

The bone screw may include an exterior and an interior. The interior may reside at least partially within the exterior and may selectively expand an expandable element of the exterior of the bone screw. More specifically, the exterior of the bone screw may include a head, a proximal portion, an intermediate portion, and a distal portion. The interior of the bone screw may include a head, an elongated element, and a distal portion.

The head of the bone screw may be enlarged relative to a remainder of the bone screw. The head may be engaged by a tool that introduces the bone screw into bone. The head may also enable assembly of the bone screw with other hardware (e.g., a rod, a plate, etc.).

The proximal portion of the exterior of the bone screw may extend distally from the head. The proximal portion may include proximal threads, which may facilitate introduction of the bone screw into bone and anchor the bone screw in the bone. The intermediate portion of the bone screw may be distally adjacent to the proximal portion, or adjacent to a distal side of the proximal portion.

The intermediate portion of the exterior of the bone screw be distally adjacent to the proximal portion of the exterior, or it may be located on a distal side of the proximal portion. A length of the intermediate portion may be compressible (i.e., axially compressible, longitudinally compressible), or the length of the intermediate element may be selectively shortened (and optionally re-lengthened). The intermediate portion of the exterior of the bone screw may include the expandable element. The expandable element may comprise a tubular element that includes a plurality of slits extending along a length of the expandable element, or a length of the intermediate element. The plurality of slits may be arranged (e.g., longitudinally offset, etc.) to define a plurality of struts extending along the length of the intermediate portion. An arrangement of the slits may enable the struts to extend radially and twist when a length of the intermediate portion of the exterior of the bone screw is compressed, or shortened.

The distal portion of the exterior of the bone screw may be distally adjacent to the intermediate portion, or it may be located on a distal side of the intermediate portion. The distal portion may include distal threads, which may facilitate introduction of the bone screw into bone and anchor the bone screw in the bone. A distal tip of the distal portion may facilitate its introduction into bone (e.g., it may be pointed, include boring features, etc.).

Each of the head, proximal portion, intermediate portion, and distal portion of the exterior of the bone screw may include an interior, or a central passage. The central passages may be aligned with each other. The central passages through the head, proximal portion, and intermediate portion of the exterior may receive corresponding portions of the elongated element of the interior. The central passage through the distal portion of the exterior may receive at least a portion of the distal portion of the interior of the bone screw.

The head of the interior of the bone screw may be accessible from the head of the exterior of the bone screw. In some embodiments, the head of the interior may be positionable or positioned within a receptacle of the head of the exterior. In other embodiments, the head of the interior may protrude from the head of the exterior.

The elongated element of the interior of the bone screw may extend through central passages, or interiors, of the head, proximal portion, and intermediate portion of the exterior. The elongated element may be free to move longitudinally through the aligned central passages and/or rotate within the aligned central passages.

The distal portion of the interior of the bone screw may be continuous with the elongated element. The distal portion may engage the distal portion of the exterior of the bone screw in a manner that enables the distal portion of the exterior to be selectively forced toward the proximal portion of the exterior to enable the shortening of the intermediate portion of the exterior. In some embodiments, the distal portion of the interior may include external threads that cooperate with internal threads of the distal portion of the exterior in such a way that rotation of the interior within the exterior may pull the distal portion of the exterior proximally, axially or longitudinally compressing, or shortening, the intermediate portion and expandable element of the exterior. In other embodiments, the interior may the distal portion of the interior may comprise a pull rod with a distal portion that engages the distal portion of the exterior in such a way that pulling the pull rod proximally pulls the distal portion of the exterior proximally, compressing, or shortening the intermediate portion and expandable element of the exterior. Such a pull rod may be rotatable between a locked position that fixes a length of the exterior and an unlocked position that enables the length of the exterior to be shortened or lengthened.

A bone screw of this disclosure may be configured as a pedicle screw, which may include an exterior with a head that includes a receptacle for a rod.

In another aspect, methods for using bone screws are disclosed. Such a method may include screwing the bone screw into a bone and axially or longitudinally compressing an intermediate portion of an exterior of the bone screw. Compression of the intermediate portion may include forcing a distal side of the intermediate portion proximally while maintaining a position of a proximal side of the intermediate portion. Such compression may be achieved by rotating an internal element of a bone screw and/or pulling an internal element of a bone screw proximally. As the intermediate portion and an expandable element of the intermediate portion are compressed, struts of the expandable element may be forced radially (i.e., compression) and twisted (i.e., torsion).

Other aspects of the disclosed subject matter, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

In the drawings:

FIG. 1 is an isometric view of an embodiment of a bone screw that may expand radially;

FIG. 2 is an isometric view of an embodiment of an assembly that includes the bone screw of FIG. 1 and an optional pedicle head;

FIG. 3 is a side view of the embodiment of the assembly shown in FIG. 2, with an expandable element of an intermediate portion of an exterior of the bone screw in an expanded arrangement;

FIG. 4 is a proximal end view of the embodiment of the assembly shown FIG. 2 with the expandable section in the arrangement shown in FIG. 3;

FIG. 5 is an isometric view of the embodiment of the assembly shown in FIG. 2 with the expandable section in the arrangement shown in FIG. 3;

FIG. 6 is another isometric view of the embodiment of the assembly shown in FIG. 2 with the expandable section in the arrangement shown in FIG. 3;

FIG. 7 is a cross-sectional representation of the embodiment of the bone screw shown in FIG. 1;

FIGS. 8-10 are side views of various embodiments of expandable elements of bone screws;

FIG. 11 is an isometric view of another embodiment of a bone screw, depicting the bone screw in an uncompressed arrangement;

FIG. 12 is an isometric view of the embodiment of the bone screw shown in FIG. 11, depicting the bone screw in a compressed arrangement;

FIG. 13 is an isometric view of yet another embodiment of a bone screw, depicting the bone screw in an uncompressed arrangement;

FIG. 14 is an isometric view of the embodiment of the bone screw shown in FIG. 13, depicting the bone screw in a compressed arrangement;

FIG. 15 is an isometric view of yet another embodiment of a bone screw, depicting the bone screw in an uncompressed arrangement; and

FIG. 16 is an isometric view of the embodiment of the bone screw shown in FIG. 15, depicting the bone screw in a compressed arrangement.

### DETAILED DESCRIPTION

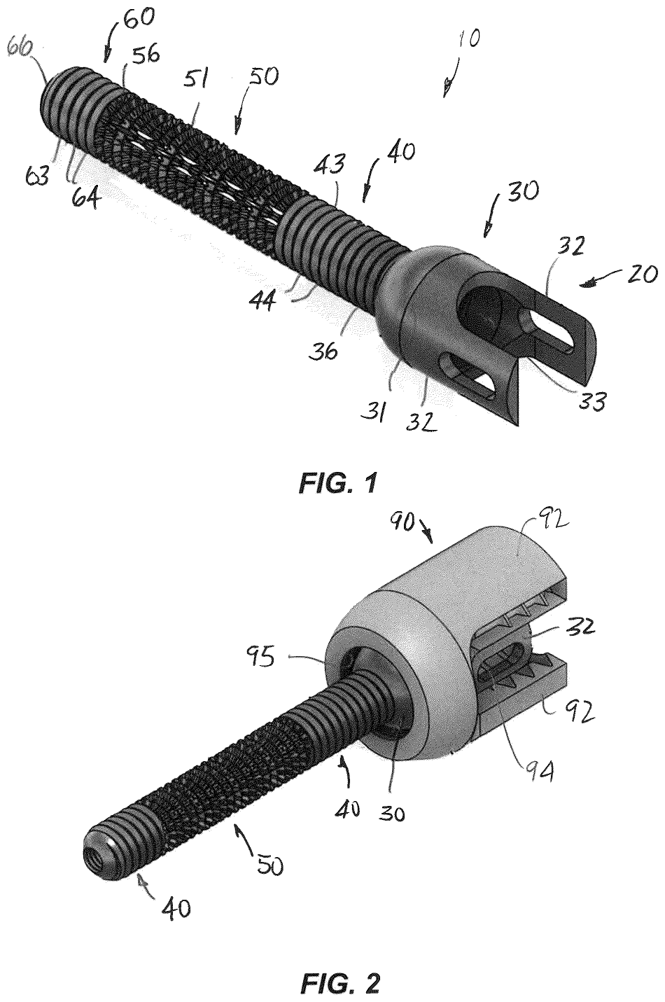

FIGS. 1-7 illustrate an embodiment of a bone screw 10, which may also be referred to as an orthopedic screw, an orthopedic bolt, or, more simply, as a screw or a bolt. More specifically, FIG. 1 shows an exterior 20 of the bone screw 10. The exterior 20 of the bone screw 10 includes a head 30, a proximal portion 40, an intermediate portion 50, and a distal portion 60.

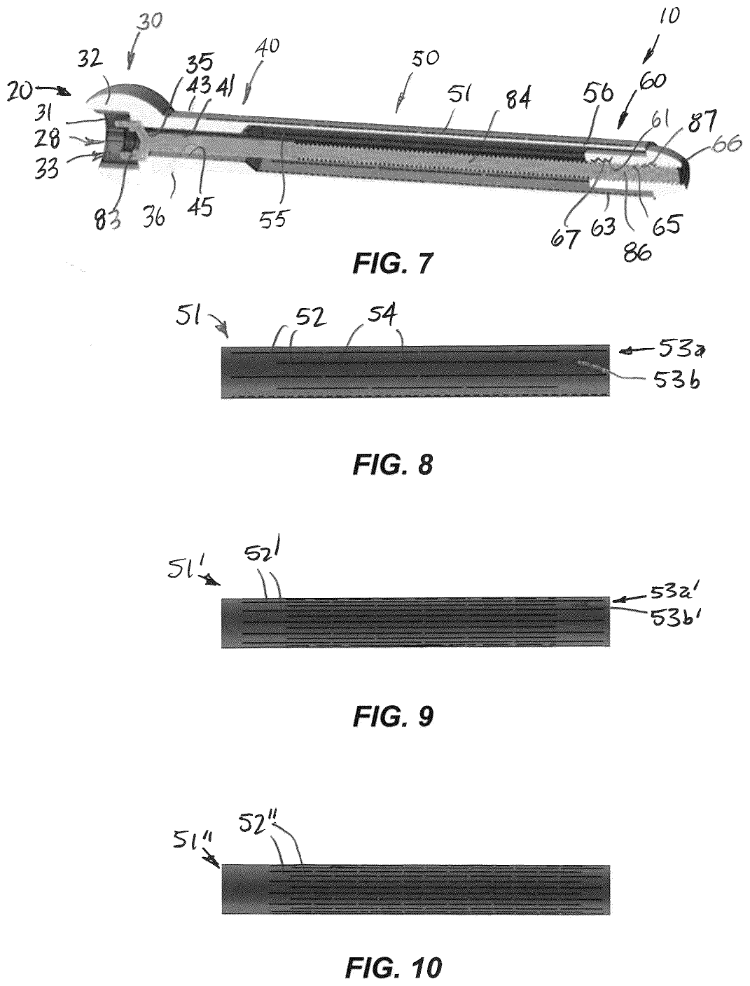

The head 30 of the exterior 20 of the bone screw 10 may have a configuration that enables the head 30 to be engaged by a tool, such as a drill, that may be used to introduce the bone screw 10 into bone. As depicted, a pair of arms 32 that are diametrically opposed to one another extend from a proximal side 31 of the head 30 includes. A receptacle 33 is also defined in the proximal side 31 of the head 30 and may be accessible between the arms 32. As shown in FIG. 7, a central passage 35 communicates with the receptacle 33 and extends through the head 30.

The proximal portion 40 of the exterior 20 of the bone screw 10 extends distally from a distal side 36 of the head 30. The proximal portion 40 is cylindrical in shape and includes an interior 41 and an exterior 43. As shown in FIG. 7, the interior 41 defines a central passage 45 through the length of the proximal portion 40. The exterior 43 may carry helical threads 44, which may facilitate introduction of the proximal portion 40 into bone and anchor the proximal portion 40 to the bone.

The intermediate portion 50 of the exterior 20 of the bone screw 10 extends distally from a distal end 46 of the proximal portion 40. The intermediate portion 50 comprises an expandable element 51, which may be tubular in shape. More specifically, the expandable element 51 may be tubular. Even more specifically, the expandable element may comprise a hypotube. In some embodiments, the hypotube may be formed from a metal or a metal alloy. For example, the hypotube may be defined from a stainless steel (e.g., an austenitic stainless steel, such as grade 304 stainless steel, grade 316 stainless steel, grade 316L stainless steel, etc.). As another example, the hypotube may be defined from titanium.

As shown in FIG. 8, the expandable element 51 may be defined by forming a plurality of cuts, or slits 52, through the tube. Without limitation, the slits 52 may be formed by laser cutting the tube. The slits 52 may be oriented parallel to one another and extend at least partially along a length of the tube (e.g., longitudinally, helically, etc.). More specifically, the slits 52 may be oriented parallel to one another and in rows 53a and 53b in which slits 52 are arranged end-to-end. The slits 52 of a row 53a, 53b of slits 52 may be offset from the slits 52 of each circumferentially adjacent row 53b, 53a of slits 52. The first rows 53a′ and second rows 53b′ may alternate with each other around a circumference of the tube. The offsets may be arranged as a so-called “running bond pattern” of slits 52, in which each slit 52 extends along about half of a length of each circumferentially adjacent slit 52. Struts 54 may be defined between circumferentially adjacent rows 53a and 53b of slits 52.

FIG. 9 illustrates an embodiment of an intermediate element 51′ in which circumferentially adjacent rows 53a′ and 53b′ of slits 52′ are positioned closer to each other than the arrangement of slits 52 shown in FIG. 8. FIG. 10 illustrates an embodiment of an intermediate element 51″ in which the slits 52″ are shorter than the slits 52 and 52′ shown in FIGS. 8 and 9, respectively. Other arrangements of slits are also within the scope of this disclosure.

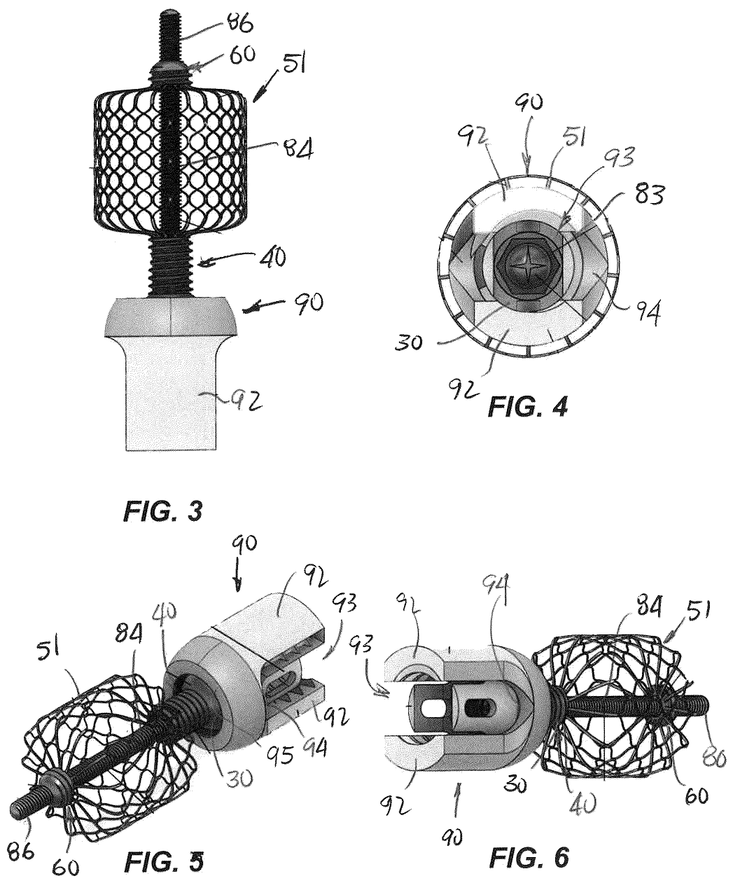

The expandable element 51 has an unexpanded arrangement, as shown in FIGS. 1 and 8, and an expanded arrangement, as shown in FIGS. 3-6 and 8. The unexpanded arrangement may also be referred to as an uncompressed arrangement, while the expanded arrangement may also be referred to as a compressed arrangement.

As shown in FIG. 7, a central passage 55 extends through the length of the intermediate portion 50 of the exterior 20 of the bone screw 10.

With returned reference to FIGS. 1-7, the distal portion 60 of the exterior 20 of the bone screw 10 extends distally from a distal end 56 of the intermediate portion 50. The distal portion 60 is cylindrical in shape. The distal portion 60 includes a distal tip 66. The distal tip 66 may have a shape that facilitates introduction of the exterior 20 of the bone screw 10 into bone. Optionally, the distal portion 60 may include an inner surface 61. As shown in FIG. 7, the optional inner surface 61 may define a central passage 65 through part of the length of the distal portion 60. The distal portion 60 also includes an exterior 63. The exterior 63 may carry helical threads 64, which may facilitate introduction of the distal portion 60 into bone and, optionally, anchor the distal portion 60 to the bone.

With continued reference to FIG. 7, an interior 80 of the bone screw 20 is shown. The interior 80 of the bone screw 10 includes a head 83, an elongated element 84, and a distal portion 86.

The head 83 of the interior 80 of the bone screw 10 may be received by the receptacle 33 in the proximal side 32 of the head 30 of the exterior 20 of the bone screw 10. The head 83 may have a configuration that enables it to be engaged and moved (e.g., rotated, pushed, etc.) by an individual's hand or by appropriate tool.

The elongated element 84 of the interior 80 of the bone screw 10 may extend distally from the head 83. The elongated element 84 may be positioned within the central passages 35, 45, and 55 of the head 30, proximal portion 40, and intermediate portion 50, respectively, of the exterior 20 of the bone screw 10.

The distal portion 86 of the interior 80 of the bone screw 10 may be continuous with the elongated element 84. The distal portion 86 may engage the distal portion 60 of the exterior 20 of the bone screw 10. In the embodiment illustrated by FIG. 7, external threads 87 on the distal portion 86 of the interior 80 may cooperate with internal threads 67 within the central passage 65 of the distal portion 60. With such an arrangement, the interior 80 of the bone screw 10 may be rotated relative to the exterior 20 of the bone screw 10 to pull the distal portion 60 of the exterior 20 proximally against the distal end 56 of the intermediate portion 50 of the exterior.

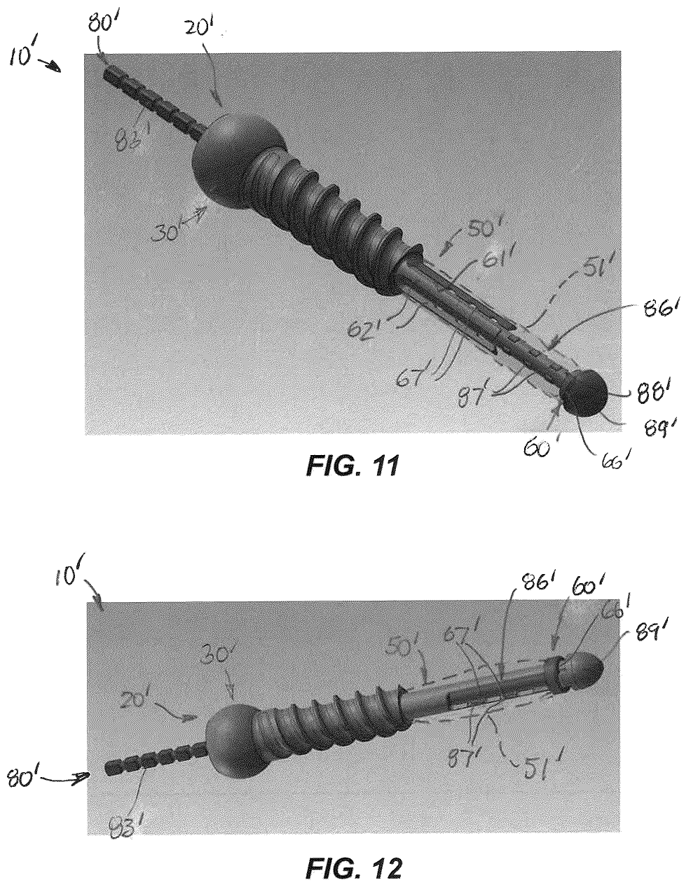

FIGS. 11 and 12 depict an embodiment of a bone screw 10′ with an interior 80′ that comprises a pull rod. The distal portion 86′ includes an enlarged distal end 89′ that extends beyond and abuts a distal end 66′ of the distal portion 60′ of the exterior 20′ of the bone screw 10′. Thus, as the interior 80′ of the bone screw 10′ is pulled proximally, it causes the enlarged distal end 89′ to force the distal portion 60′ proximally against the expandable element 51′, axially or longitudinally compressing the expandable element 51′.

In addition, the distal portion 86′ of the interior 86′ includes rows 88′ of teeth 87′. The teeth 87′ in each row 88′ are longitudinally spaced apart from each other. The rows 88′ are circumferentially spaced apart from each other. For example, as depicted, there may be four rows 88′ or any other suitable number of rows 88′ (e.g., one row 88′, two rows 88′, three rows 88′, etc.). The spacing of the rows 88′ corresponds to the circumferential spacing between separate, longitudinally extending segments 62′ of the intermediate portion 50′ and/or distal portion 60′ of the exterior 20′ of the bone screw 10′. For example, as depicted, there may be four segments 62′ or any other suitable number of segments 62′ (e.g., one segment 62′ with a longitudinal slot in it, two segments 62′, three segments 62′, etc.). The spacing of the teeth 87′ corresponds to spacing of recesses 67′ within the inner surfaces 61′ of the segments 62′.

Thus, when the interior 80′ is rotated to align the rows 88′ with the spaces between circumferentially adjacent segments 62′, as shown in FIG. 11, the interior 80′ may be pulled, with the teeth 87′ sliding longitudinally between the circumferentially adjacent segments 62′. When the interior 80′ and its distal portion 86′ have been pulled to a desired position, they may again be rotated (e.g., a quarter turn, or about 90°, etc.) to move the teeth 87′ into corresponding recesses 67′ and, thus, to lock the distal portion 86′ of the interior 80′ into place to maintain the shortened length of the bone screw 10′, as shown in FIG. 12, and the compressed arrangement, or expanded arrangement, of the expandable element 51′. A proximal end, or a head 83′, of the interior 80′ may then be cut or broken so that it does not extend beyond the head 30′ of the exterior 20′.

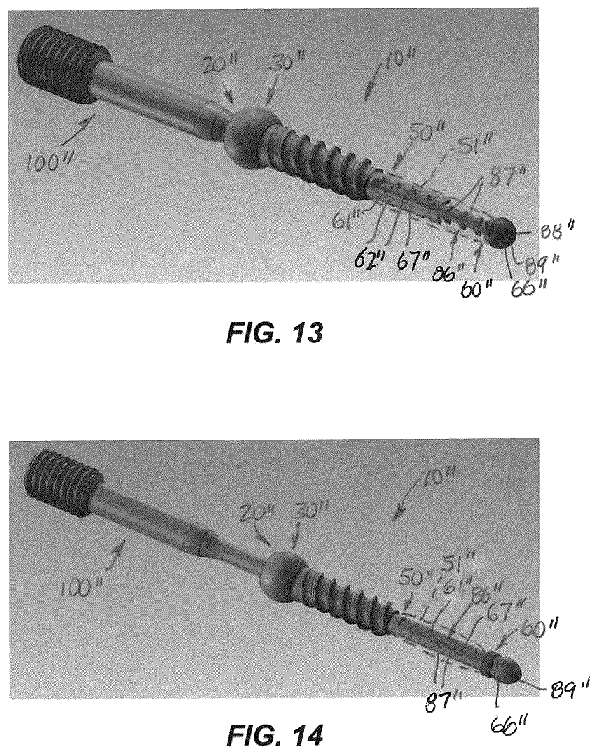

FIGS. 13 and 14 also depict an embodiment of a bone screw 10″ with an interior (not shown) that comprises a pull rod. The head (not shown) of the interior, which is located within a receptacle in the head 30″ of the exterior 20″ of the bone screw 10″ is configured to be coupled to a tool 100″ that extends proximally beyond the head 30″. The distal portion 86″ of the interior includes an enlarged distal end 89″ that extends beyond and abuts a distal end 66″ of the distal portion 60″ of the exterior 20″ of the bone screw 10″. Thus, as the tool 100″ and the interior of the bone screw 10″ are pulled proximally, they cause the enlarged distal end 89″ to force the distal portion 60″ proximally against the expandable element 51″, compressing the expandable element 51″.

In addition, the distal portion 86″ of the interior includes rows 88″ of teeth 87″. The teeth 87″ in each row 88″ are longitudinally spaced apart from each other. The rows 88″ are circumferentially spaced apart from each other. For example, as depicted, there may be four rows 88″ or any other suitable number of rows 88″ (e.g., one row 88″, two rows 88″, three rows 88″, etc.). The spacing of the rows 88″ corresponds to the circumferential spacing between separate, longitudinally extending segments 62″ of the distal portion 60″ and/or intermediate portion 50″ of the exterior 20″ of the bone screw 10″. For example, as depicted, there may be four segments 62″ or any other suitable number of segments 62″ (e.g., one segment 62″ with a longitudinal slot in it, two segments 62″, three segments 62″, etc.). The spacing of the teeth 87″ corresponds to spacing of notches 67″ within an edge 61″ of each segment 62″.

Thus, when the tool 100″ and the interior are rotated to align the rows 88″ with the spaces between circumferentially adjacent segments 62″, as shown in FIG. 13, the tool 100″ may be pulled, with the teeth 87″ sliding longitudinally between the circumferentially adjacent segments 62″. When the distal portion 86″ has been pulled to a desired position, the tool 100″ may again be rotated (e.g., a quarter turn, or about 90°, etc.) to move the teeth 87″ into corresponding notches 67″ and, thus, to lock the distal portion 86″ into place to maintain the shortened length of the bone screw 10″, as shown in FIG. 14, and the compressed arrangement, or expanded arrangement, of the expandable element 51″. The tool 100″ may then be uncoupled from the interior of the bone screw 10″.

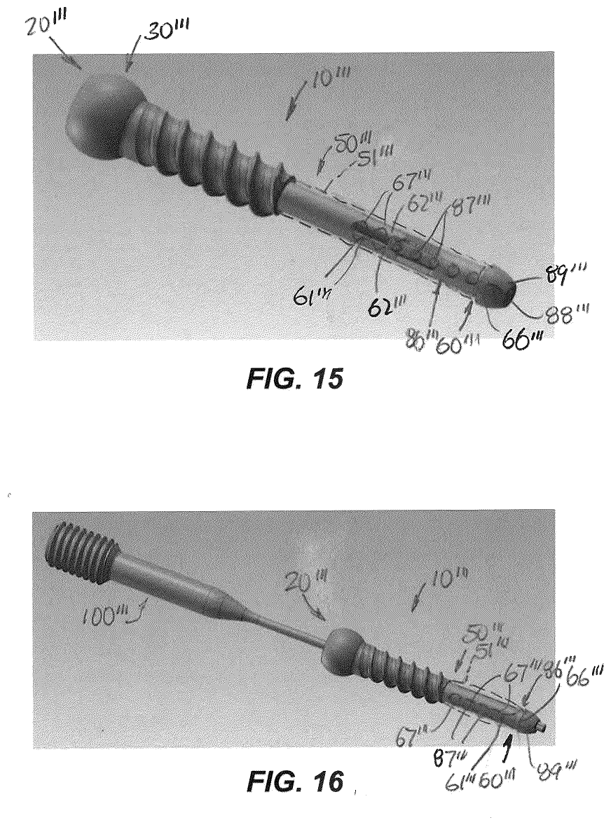

FIGS. 15 and 16 depict another embodiment of a bone screw 10″ with an interior (not shown) that comprises a pull rod. The head (not shown) of the interior, which is located within a receptacle in the head 30′″ of the exterior 20″ of the bone screw 10′″ is configured to be coupled to a tool 100′″ that extends proximally beyond the head 30′″. The distal portion 86′″ of the interior includes an enlarged distal end 89″ that extends beyond and abuts a distal end 66″ of the distal portion 60′″ of the exterior 20′″ of the bone screw 10′″. Thus, as the tool 100′″ and the interior of the bone screw 10′″ are pulled proximally, they cause the enlarged distal end 89′″ to force the distal portion 60′″ proximally against the expandable element 51′″, compressing the expandable element 51′″.

In addition, the distal portion 86′″ of the interior includes rows 88′″ of round protrusions 87′″. The round protrusions 87′″ in each row 88′″ are longitudinally spaced apart from each other. The rows 88′″ are circumferentially spaced apart from each other. For example, as depicted, there may be four rows 88′″ or any other suitable number of rows 88′″ (e.g., one row 88′″, two rows 88′″, three rows 88′″, etc.). The spacing of the rows 88′″ corresponds to the circumferential spacing between separate, longitudinally extending segments 62′″ of the distal portion 60′″ and/or intermediate portion 50′″ of the exterior 20″ of the bone screw 10′″. For example, as depicted, there may be four segments 62′″ or any other suitable number of segments 62′″ (e.g., one segment 62′″ with a longitudinal slot in it, two segments 62′″, three segments 62′″, etc.). The spacing of the round protrusions 87′″ corresponds to spacing of notches 67′″ within opposed edges 61′″ of circumferentially adjacent segments 62′″.

Thus, when the tool 100′″ and the interior are pulled, the round protrusions 87′″ may slide longitudinally between the circumferentially adjacent segments 62′″ and be pulled, or snapped, into place between pairs of opposed notches 67′″ on the opposed edges 61′″ of the circumferentially adjacent segments 62″, as shown in FIG. 16. When the distal portion 86′″ has been pulled to a desired position, the opposed pairs of notches 67′″ may lock the distal portion 86″ into place to maintain the shortened length of the bone screw 10′″ and the compressed arrangement, or expanded arrangement, of the expandable element 51′″. The tool 100′″ may then be uncoupled from the interior of the bone screw 10′″.

With returned reference to FIGS. 1 and 8, as the distal portion 60 moves proximally against the distal end 56 of the intermediate portion 50, the distal portion 60 may compress the expandable element 51 of the intermediate portion 50, shortening a length of the expandable element 51 and causing the struts 54 of the expandable element 51 to move apart from each other (by way of the slits 52) and to expand radially and to twist, or rotate about their longitudinal axes, placing the expandable element 51 in its compressed arrangement, or its expanded arrangement, as shown in FIGS. 3-6. When this occurs within bone, the struts 54 may press against and engage the bone, which may further anchor the bone screw 10 within the bone.

FIGS. 2-6 show an optional pedicle head 90, which may be secured over the positioned over the head 30 of the exterior 20 of the bone screw 10. More specifically, the pedicle head 90 may include a central passage 95 that receives the proximal portion 40 of the bone screw and a receptacle 93 that receives the head 30 of the bone screw 10. The head 30 of the bone screw 10 may move within the receptacle 93, which may enable the bone screw 10 to pivot relative to the pedicle head 90. Thus, the pedicle head 90 may facilitate positioning of the bone screw 10 with expanded flexibility, allowing for expanded orientations of the bone screw 10 in a vertebral body while still enabling engagement of hardware (e.g., a rod, etc.) by the pedicle head 90. More specifically, hardware (e.g., a rod, etc.) may be received by a contoured surface 94 between diametrically opposed arms 92 of the pedicle head 90.

As alternatives to pedicle screws, a bone screw may comprise an osseointegration screw for use with prosthetics, an orthopedic interference screw, an orthopedic locking screw, an orthopedic suture anchor, or the like.

Methods of using a bone screw 10, 10′, 10″, 10′″, etc., of this disclosure should be apparent from the foregoing description. As the expandable element 51, 51′, 51″, 51′″, etc., of the bone screw 10, 10′, 10″, 10′″, etc., is compressed and expands, it may exert forces against the bone into which it has been introduced that engage the bone and, thus, anchor the bone screw to the 10, 10′, 10″, 10′″, etc. Such anchoring may be effected regardless of whether the bone screw 10, 10′, 10″, 10′″, etc., is implanted into the bone bicortically or unicortically. The methods may be used in a variety of procedures, including without limitation, for the fixation of bony fractures, to secure hardware to bone (e.g., in joint replacement surgery, etc.), in dental surgeries (e.g., implanting teeth, etc.), and the like.

Although the disclosure provides many specifics, the specifics should not be construed as limiting the scope of any of the claims, but merely as providing illustrations of some embodiments of elements and features of the disclosed subject matter that fall within the scopes of the claims. Other embodiments of the disclosed subject matter may be devised that are also within the scopes of the claims. Accordingly, the scope of each claim is limited only by its plain language and the legal equivalents thereto.

What is claimed:

1. A bone screw, comprising:
an exterior including:
a head;
a proximal portion extending distally from the head and including proximal threads;
an intermediate portion distally adjacent to the proximal portion and including an expandable element comprising a plurality of slits extending along a length of the intermediate portion and arranged to define a plurality of struts extending along the length of the intermediate portion that extend radially and twist upon shortening of the intermediate portion; and
a distal portion distally adjacent to the intermediate portion; and
an interior, including:
a head;
an elongated element extending through interiors of the proximal portion and the intermediate portion of the exterior, and
a distal portion engaging the distal portion of the exterior in a manner that enables the distal portion of the exterior to be selectively forced toward the proximal portion of the exterior to enable the shortening of the intermediate portion of the exterior.

2. The bone screw of claim 1, wherein the interior is rotatable within the exterior to enable the distal portion of the exterior to be selectively forced toward the proximal portion of the exterior.

3. The bone screw of claim 2, wherein:
the distal portion of the exterior includes internal threads; and
the distal portion of the interior includes external threads that cooperate with the internal threads of the distal portion of the exterior.

4. The bone screw of claim 2, wherein the interior comprises a pull rod.

5. The bone screw of claim 4, wherein the pull rod is rotatable between a locked position that fixes a length of the exterior and an unlocked position that enables the length of the exterior to be shortened or lengthened.

6. The bone screw of claim 1, wherein the plurality of slits of the intermediate portion of the exterior includes:
at least one first row of first slits arranged end to end; and
at least one second row of second slits circumferentially adjacent to the at least one first row of slits, with the second slits being arranged end to end and the second slits being longitudinally offset from the first slits.

7. The bone screw of claim 1, wherein the plurality of slits are arranged parallel to a longitudinal axis of the intermediate portion of the exterior.

8. The bone screw of claim 1, comprising a pedicle screw.

9. The bone screw of claim 8, wherein the head of the exterior includes a receptacle for a rod.

10. The bone screw of claim 1, wherein the distal portion of the exterior includes distal threads.

11. A pedicle screw, comprising:
an exterior including:
a head comprising a receptacle for a rod;
a proximal portion extending distally from the head and including proximal threads;
an intermediate portion distally adjacent to the proximal portion and including an expandable element comprising a plurality of slits extending along a length of the intermediate portion and arranged to define a plurality of struts extending along the length of the intermediate portion that extend radially and twist upon shortening of the intermediate portion; and
a distal portion distally adjacent to the intermediate portion and including distal threads; and
an interior, including:
a head;
an elongated element extending through interiors of the proximal portion and the intermediate portion of the exterior, and
a distal portion engaging the distal portion of the exterior in a manner that enables the distal portion of the exterior to be selectively forced toward the proximal portion of the exterior to enable the shortening of the intermediate portion of the exterior.

12. The bone screw of claim 11, wherein the interior is rotatable within the exterior to enable the distal portion of the exterior to be selectively forced toward the proximal portion of the exterior.

13. The bone screw of claim 12, wherein:
the distal portion of the exterior includes internal threads; and
the distal portion of the interior includes external threads that cooperate with the internal threads of the distal portion of the exterior.

14. The bone screw of claim 12, wherein the interior comprises a pull rod.

15. The bone screw of claim 14, wherein the pull rod is rotatable between a locked position that fixes a length of the exterior and an unlocked position that enables the length of the exterior to be shortened or lengthened.

16. A method for using a bone screw, comprising:
screwing the bone screw into a bone; and
longitudinally compressing an intermediate portion of an exterior of the bone screw, including applying a radial force and a twisting force to struts of the intermediate portion.

17. The method of claim 16, wherein longitudinally compressing the intermediate element comprises forcing a distal end of the intermediate portion proximally.

18. The method of claim 17, wherein forcing the distal end of the intermediate portion proximally comprises rotating an internal element of the bone screw.

19. The method of claim 17, wherein forcing the distal end of the intermediate portion proximally comprises pulling an internal element of the bone screw proximally.

20. The method of claim 16, wherein screwing the bone screw into the bone comprises unicortically screwing the bone screw into the bone.
